(19) United States
(12) Patent Application Publication
(10) Pub. No.: US 2006/0229671 A1
(43) Pub. Date: Oct. 12, 2006

(54) Suture anchor and suture anchor installation tool

(75) Inventors: Anton J. Steiner, Wharton, NJ (US); Cesar D. Chavista, Jersey City, NJ (US); David I. Knight, Perth Amboy, NJ (US); James Shock, Glen Rock, NJ (US)

Correspondence Address: JOHN S. HALE;GIPPLE & HALE, 6665-A OLD DOMINION DRIVE, MCLEAN, VA, 22101, US

(73) Assignee: Musculoskeletal Transplant Foundation

(21) Appl. No.: 11/101,569

(22) Filed: Apr. 8, 2005

(51) Int. Cl. A61B 17/04

(52) U.S. Cl. 606/232

(57) ABSTRACT

A sterile suture anchor comprising a body with a distal end, tapered planar side walls and a dome shaped proximal end, a cross section of said body forming a triangular shape with at least one rounded end; and a suture aperture transversely cut through the body being dimensioned to hold at least one suture. An insertion tool for inserting the suture anchor through a substantially cylindrical bore hole in a live human bone and causing the suture anchor to be anchored comprises a handle, a hollow tube secured to the handle and a driver rod slidably mounted in the tube. The driver rod has a distal end defining an angled cam which engages a suture anchor mounted in said tube and rotates the suture anchor into a desired position for insertion into human tissue. A finger driver assembly is mounted on the handle and is secured to the driver and so that movement of the finger driver assembly causes the driver rod to be moved within the hollow tube causing the driver rod cam end to engage the suture anchor and turn the suture anchor in a predetermined orientation for anchoring in the bore hole.

### RELATED APPLICATIONS

There are no related applications.

### BACKGROUND OF THE INVENTION

1. Field of Invention

The field of art to which this invention relates is generally directed to suture anchors and associated suture anchor installation tools and more specifically to a suture anchor constructed of allograft bone which is oriented by the installation tool into a specific orientation within a bore cut into a human bone to secure a suture in body tissue.

2. Description of the Prior Art

As the treatment of injuries to joints and soft tissue has progressed, a need has developed for medical devices which can be used to attach tendons, ligaments and other soft tissue to bone. When surgically repairing an injured joint, it is preferable to restore the joint by reattaching the damaged soft tissues such as ligaments and tendons to bone rather than replacing them with an artificial material. An increase in the incidence of injuries to joints involving soft tissue has been observed. This increased incidence may be due, at least in part, to an increase in participation by the public in various physical activities such as sports and other recreational activities. These types of activities may increase the loads and stress placed upon joints, sometimes resulting in joint injuries with corresponding damage to associated soft tissue. There are well over 500,000 surgical procedures performed in the United States annually in which soft tissue was attached to a bone in various joints including the shoulder, hip and knee.

One conventional orthopedic procedure for reattaching soft tissue to bone is performed by initially drilling holes or tunnels at predetermined locations through a bone in the vicinity of a joint. The surgeon approximates soft tissue to the surface of the bone using sutures threaded through these holes or tunnels. This method is a time consuming procedure resulting in the generation of numerous bone tunnels. The bone tunnels, which are open to various body fluids and infectious agents, may become infected or break and complications such as a longer bone-healing period may result. One known complication of drilling tunnels across bone is that nerves and other soft tissue structures may be injured by the drill bit or orthopaedic pin as it exits the far side of the bone. Also, it may be anatomically impossible or at least very difficult to reach and/or secure a suture that has been passed through a tunnel. When securing the suture or wire on the far side of the bone, nerves and soft tissues can become entrapped and damaged.

Screws are also used to secure soft tissues adjacent to the bone surface. Screws suffer from the disadvantage that they tend to loosen with time, thereby requiring a second operation to remove the loosened screw. In addition, when the screws are set in bone, the heads of the screws frequently protrude above the surface of the bone in which they are set, thereby presenting an abrasive surface which may create wear problems with surrounding tissue. Once a hole has been made in the bone it may be impossible to relocate the hole a small distance away from its original position due to the disruption of the bone structure created by the initial hole. Finally, the nature of a screw attachment tends to require a flat attachment geometry; namely that the pilot hole must generally be located on a relatively flat section of the bone, and toothed washers must frequently be used in conjunction with the screws to fasten the desired objects to the target bone. As a result of these constraints, it may be necessary to locate the attachment point at less than an optimal position.

Staples are also used to secure soft tissue adjacent the bone surface. Staples suffer from their own set of disadvantages and must frequently be removed after they have been in position for some time, thereby necessitating a second operation. In addition, staples must generally be positioned so as to maximize their holding power in the bone which may conflict with the otherwise-optimal position for attachment of the objects to bone. Staples have also been known to crack the bone during deployment, or to accidentally transect the object (e.g. soft tissue) being attached to the bone, since it tends to be difficult to precisely control the extent of the staple's penetration into the bone. Additionally, once the staple has been set into the bone, the position of the staple is then effectively determined, thereby making it impossible to later adjust the position of the staple or to adjust the degree of tension being applied to the object which is being attached to the bone without using a new staple.

In order to overcome a number of the problems associated with the use of the conventional soft tissue to bone attachment procedures, suture anchors have been developed and are now frequently used to attach soft tissue to bone. A suture anchor, commonly referred to as a bone anchor, is an orthopedic, medical device which is typically implanted into a cavity drilled into a bone. In the present application, the device will be referred to as a suture anchor. The bone cavity is typically referred to as a bore hole and if it does not extend through the bone, it is typically referred to as a “blind hole”. The bore hole is typically drilled through the outer cortical layer of the bone and into the inner cancellous layer. The suture anchor may be engaged in the bore hole by a variety of mechanisms including friction fit barbs which are forced into the cancellous layer of bone or by threading into pre-threaded bores in the bone mass or by using self tapping threads. Suture anchors have many advantages including reduced bone trauma, simplified application procedures, and decreased likelihood of suture failure. Suture anchors may be used in shoulder reconstruction for repairing the glenohumeral ligament and may also be used in surgical procedures involving rotator cuff repair, ankle and wrist repair, bladder neck suspension, and hip replacement.

Suture anchors typically have a hole or opening cut therein for receiving a suture. The suture extends out from the anchor and bore hole and is used to attach soft tissue. The suture anchors presently described in the art may be made of absorbable materials which absorb over time, or they may be made from various non-absorbable, biocompatible materials. Although most suture anchors described in the art are made from non-absorbable materials, the use of absorbable suture anchors may result in fewer complications since the suture anchor is absorbed and replaced by bone over time. The use of absorbable suture anchors may reduce the likelihood of damage to local joints caused by anchor migration. Moreover, when an absorbable suture anchor is fully absorbed it will no longer be present as a foreign body. It is also advantageous to construct the bone anchor out of allograft cortical bone as this material will result in natural filling in of the bore with bone in the original bone base and the elimination of foreign material from the site.

It is desirable that a suture anchor be of a small size to minimize damage to the tissue in which the suture is anchored and that the suture anchor be easily attached and hold the suture firmly in place. A number of different suture anchors exist in the prior art including a barb-type anchor, a threaded or screw type anchor, and insert type anchor and a wedge type anchor.

In practice, wedge type anchors with attached sutures are introduced with an insertion tool into a bore drilled into the bone at the location where the body tissue is to be attached. The suture anchor generally includes a first gripping portion in the form of a sharp edge or a point and is designed to rotate as it enters the bore or is in the process of being withdrawn from the bore. As the suture anchor rotates the first gripping portion penetrates the cancellous wall of the bore and causes further rotation of the anchor. At the opposite end of the suture anchor, a second gripping portion is provided which penetrates into the cancellous wall of the bore opposite the first gripping portion as the suture anchor is rotated wedging the suture anchor within the bore in a seated position where it is anchored in the bone and ready to have adjacent body tissue attached thereto.

U.S. Pat. No. 5,683,418 issued Nov. 4, 1997 is directed toward an extruded or injection molded suture anchor which provides an offset force to the suture. The insert is constructed of bioabsorbable polymer material or a non-absorbable material such as metal. The suture anchor as shown in FIGS. 13-14 is in a truncated four sided wedge shape with a planar distal and proximal end and at least one rounded side. The suture anchor is formed with a throughgoing suture anchor located adjacent the distal end, the distal end having a smaller surface area than the proximal end. The suture anchor is attached to a frangible insertion rod with the smaller area distal end being pushed down into the bore so that when the proximal larger end is pulled toward the bore entrance by pulling on the suture, the sharp edges of the comers of the proximal end dig into the cancellous bone holding the suture anchor in position.

U.S. Pat. No. 6,306,158 issued Oct. 23, 2001 is directed toward a generally quadrilateral shaped body constructed of a bioabsorbable material such as polylactic acid, polydioxanone, polyglycolic acid and similar materials. The suture anchor may also be constructed of cortical bone where the bone is autologous or autogenous bone. The body of the anchor has a flat bore-abutting surface with a leading gripping edge at one end of the bore-abutting surface defined by a inclined planar side which intersects the planar abutting surface at an acute angle and a trailing gripping edge at the other end defined by an opposing planar side which intersects the planar abutting surface at an acute angle. The leading edge and the trailing edge are connected by the closing surface which has an arcuate portion and a linear portion engaging the leading and trailing gripping edges opposite the bone-abutting surface. The body defines a transverse bore and a bore to receive an insertion anchor. A second divisional U.S. Pat. No. 6,635,074 issued Oct. 21, 2003 having an identical specification and drawings to the '158 patent is directed toward a kit for anchoring a suture in a bore.

U.S. Pat. No. 5,540,718 issued Jul. 30, 1996 is directed toward a conical suture anchor having a bore in which an end of an insertion tool is inserted. When the suture anchor is placed within cancellous bone tissue, the shape memory of the insertion tool urges the suture anchor to its original position so that the suture anchor cannot fit through the bone hole thereby anchoring the same in the human bone.

U.S. Pat. No. RE 36,974 issued Nov. 28, 2000 reissued from U.S. Pat. No. 5,496,348 discloses an anchor with a tubular body having a width less than its length for securing a suture in the body. The anchor includes a tubular wall having a central axis with both ends free of axially inwardly extending slots and an inner surface extending for the entire length of the tube which defines a central opening extending between the proximal end and the distal end and a plurality of transverse throughgoing apertures. The anchor has an anchoring orientation in the body achieved by manipulation of the distal end of the anchor by pulling on a second end portion of the suture.

The above noted '974 patent is a division of application Ser. No. 08/344,466 now U.S. Pat. No. 5,527,343 which is a division of application Ser. No. 08/062,295 now U.S. Pat. No. 5,403,348. The disclosure, specification and drawings of both of these patents is identical to that of the '974 patent with the claims of the '343 patent being directed towards a method for insertion of a suture anchor and the claims of the '348 patent being directed toward an apparatus.

U.S. Pat. No. 4,899,743 issued Feb. 13, 1990 discloses a suture anchor and installation tool which comprises an elongated member having a first end and a second end and a slot extending from the first end towards the second end, the slot being sized to accommodate the suture anchor's barb. The suture anchor is cylindrical with a barb affixed to the distal end and an inclined distal end surface disposed at an angle of approximately 30 degrees to the suture anchor's longitudinal axis. A angled bore is cut through a side wall and the body of the suture anchor is placed at an angle to the face of the inclined distal end surface. When the suture anchor bottoms out in the bone hole, and the elongated member is thereafter withdrawn, the barb's engagement with the bone wall will cause the suture anchor to separate from the elongated member, leaving the suture anchor (and its attached suture) anchored securely in the bone.

Another patent, U.S. Pat. No. 4,823,794 issued on Apr. 25, 1989 discloses a surgical pledget with a V shaped lead-in to a suture entrance slit extending through the pledget from a side wall to a central suture location within the pledget and means for retaining the suture at the suture hole so that the pledget may be positioned on a medial portion of a suture.

U.S. Pat. No. 5,683,418 issued on Nov. 4, 1997 discloses a suture anchor fabricated from extruded material having a throughgoing bore which provides an offset pulling force. The preferred embodiment of the suture anchor as shown in FIGS. 1-6 has a cylindrical longitudinal surface with angled abutment ends which can be tapered to an edge on each side. A transverse bore is formed through the body offset from the center of the suture anchor such that an imbalance is formed in the rotation of the device on implantation.

Although suture anchors for attaching soft tissue to bone are available for use by the orthopedic surgeon, there is a need in this art for novel suture anchors having improved performance characteristics, such as ease of insertion and greater resistance to “pull-out”.

### SUMMARY OF THE INVENTION

The present invention is directed toward a suture anchor constructed of animal bone preferably cortical allograft human bone which has a first distal end with a rounded end wall, angled planar side walls intersecting said first distal end forming sharp end surfaces therewith, the opposite ends of the side walls ending in a dome shaped proximal end forming a generally triangular cross section. The first distal end has a length which is greater than the length of the outer surface of the proximal end with the length of the distal end also being greater than the diameter of the bore hole in which it is to be placed. A throughgoing bore is formed in the body of the suture anchor to hold suture strands and the suture body is mounted within an outer tube lumen of a driver instrument. The driver instrument has a driver rod with a camming tip which is extending forward by the surgeon to orient the suture anchor into a desired orientation as the suture anchor is transported in the outer tube lumen.

It is thus an object of the present invention to provide a suture anchor which can be formed in a very small size.

Therefore, it is another object of the present invention to provide a suture anchor which is simple to apply and is mechanically stable when implanted in bone tissue.

It is still another object of the invention to provide an improved bone anchor installation tool which is easy to manufacture and easy to use.

It is yet another object of the invention to provide an improved bone anchor installation tool whereby the bone anchor is precisely oriented in a predetermined manner.

It is a further object of the present invention to provide an absorbable suture anchor made of allograft cortical bone.

Accordingly, one of the objects of the present invention is to provide an allograft suture anchor which promotes the use of natural bone growth in the bone bore.

Yet another object of the present invention is to provide a novel suture anchor for anchoring one end of a piece of conventional suture in bone which anchor will attach itself securely to the target area of a bone bore and which has virtually no tendency to migrate from its deployment site.

Still another object of the present invention is to provide a novel suture anchor for anchoring one end of a piece of conventional suture in bone which has high tissue acceptability, prevents back out and is reliable in use.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 is a perspective view of the inventive bone suture anchor;

FIG. 2 is a top plan view of the bone suture anchor shown in FIG. 1;

FIG. 3 is a cross section of the bone suture anchor taken across line 3′-3′ of FIG. 2;

FIG. 4 is a top plan view of the inventive suture anchor insertion instrument;

FIG. 5 is a cross section view of the suture anchor insertion instrument taken along line 5′-5′ of FIG. 4;

FIG. 6 is an enlarged view of the area of FIG. 5 shown by circle A;

FIG. 7 is a top plan view of the handle of the suture anchor insertion instrument;

FIG. 8 is a cross sectional view of the handle of FIG. 7 taken along lines 8′-8′;

FIG. 9 is a side elevational view of the handle shown in FIG. 7;

FIG. 10 is a front elevational view of the distal end of the handle shown in FIGS. 7 and 9;

FIG. 11 is a cross sectional view of the handle of FIG. 9 taken along lines 11′-11′;

FIG. 12 is a cross sectional view of the handle of FIG. 9 taken along lines 12′-12′;

FIG. 13 is a cross sectional view of the handle of FIG. 9 taken along lines 13′-13′;

FIG. 14 is an enlarged view of the area of circle B taken from FIG. 7;

FIG. 15 is top plan view of the thumb activator housing;

FIG. 16 is a cross sectional view of thumb activator housing shown in FIG. 15 taken along line 16′-16′;

FIG. 17 is a front elevational view of the thumb activator housing of FIG. 15;

FIG. 18 is a bottom plan view of the thumb activator housing of FIG. 15;

FIG. 19 is a perspective view of the suture lock button;

FIG. 20 is a side elevational view of the suture lock button of FIG. 19;

FIG. 21 is a side elevational view of the suture anchor driver rod;

FIG. 22 is an enlarged cross sectional view of the driver rod of FIG. 21 taken along lines 22′-22′;

FIG. 23 is an enlarged view of the proximal end of the driver rod of FIG. 21 taken from the area shown by circle C;

FIG. 24 is the enlarged view of the distal end of the driver rod of FIG. 21 taken from the area shown by circle D;

FIG. 25 is a side elevational view of the outer tube showing the suture slot with the position of the driver rod shown in phantom;

FIG. 26 is an enlarged schematic view of the suture anchor being engaged and driven by the driver rod in the insertion tube along with the suture to show the suture anchor position;

FIG. 27 is an enlarged cross sectional sequential schematic view of the continual camming action of the driver rod on the suture anchor from the position shown in FIG. 26, the suture being omitted to show anchor movement;

FIG. 28 is an enlarged cross sectional sequential schematic view of the continual camming action of the driver rod on the suture anchor from the position shown in FIG. 27, the suture being omitted to show anchor movement;

FIG. 29 is an enlarged sequential schematic view of the continual action of the driver rod on the suture anchor from the position shown in FIG. 28, the suture being omitted to show anchor movement; and

FIG. 30 is a schematic cross sectional view of the suture anchor mounted in the bore of a bone.

### DETAILED DESCRIPTION OF THE PREFERRED EMBODIMENT

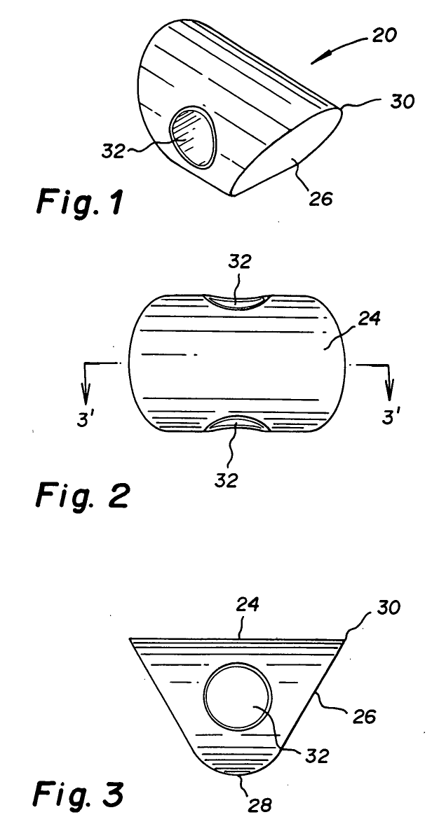

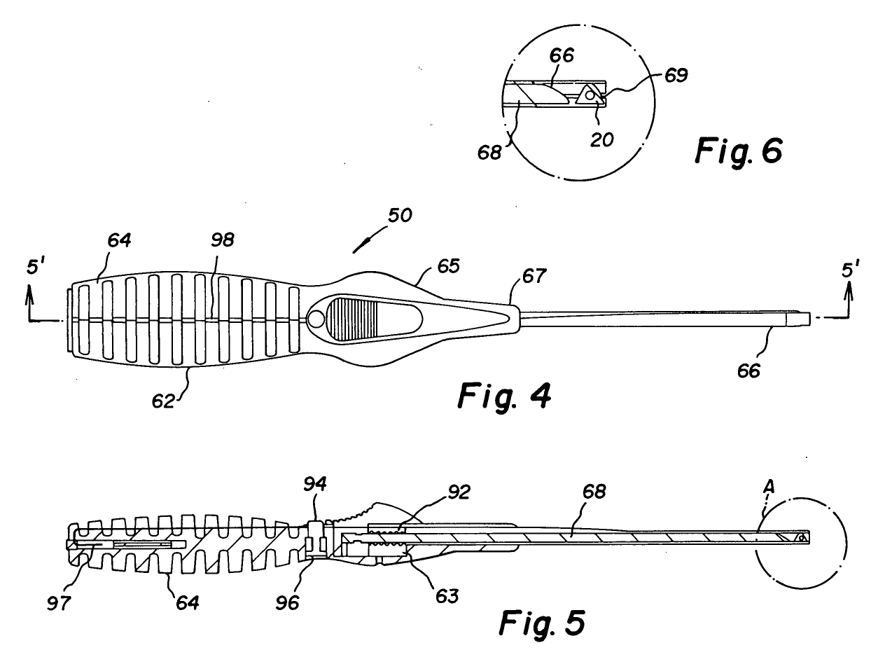

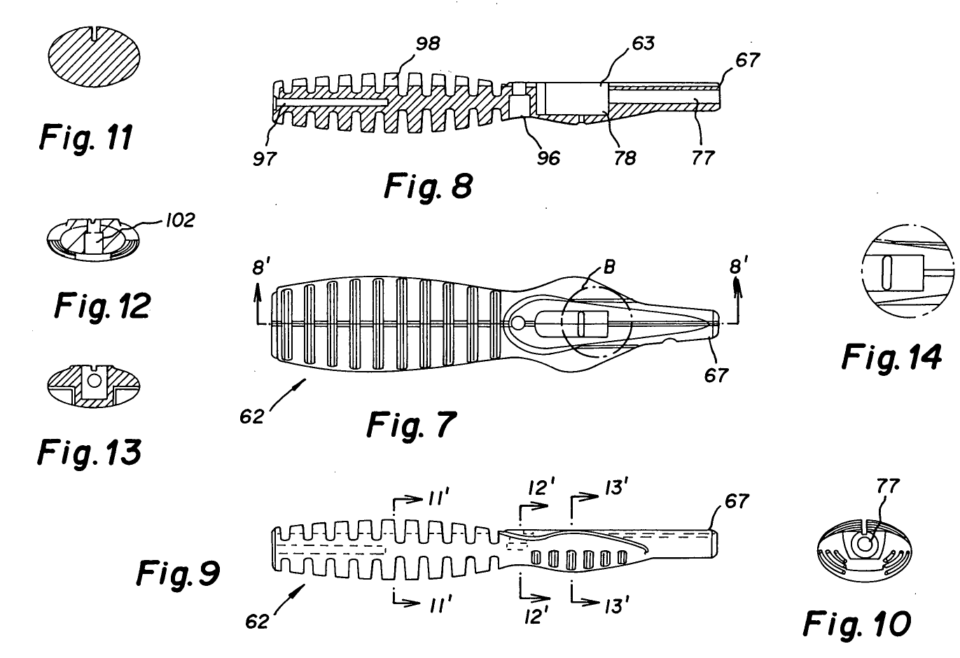

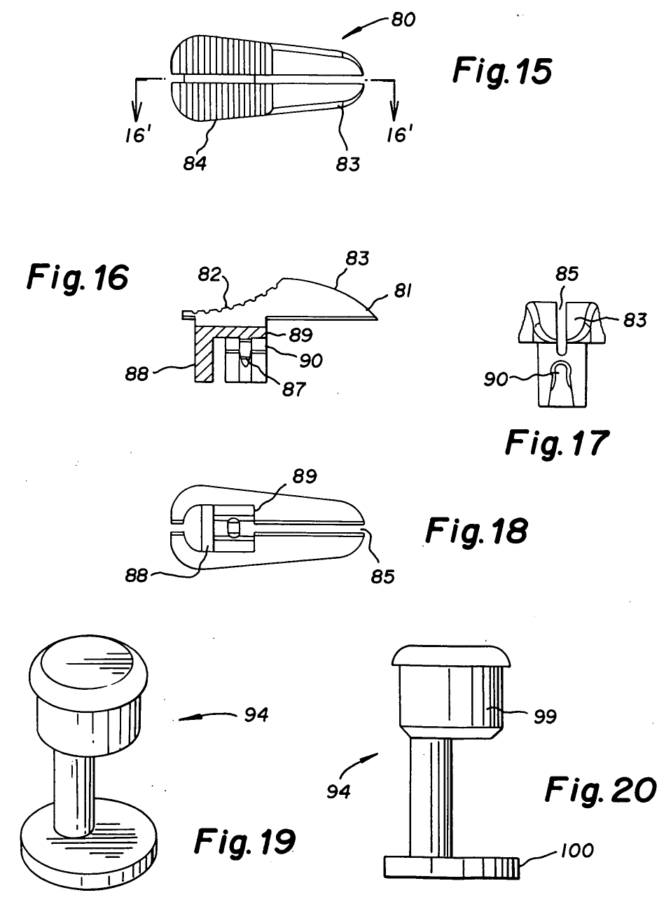

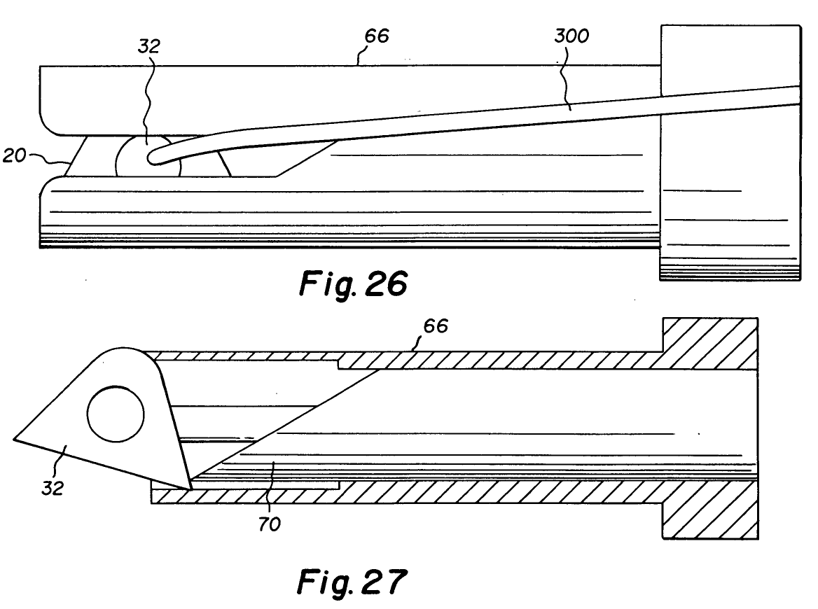

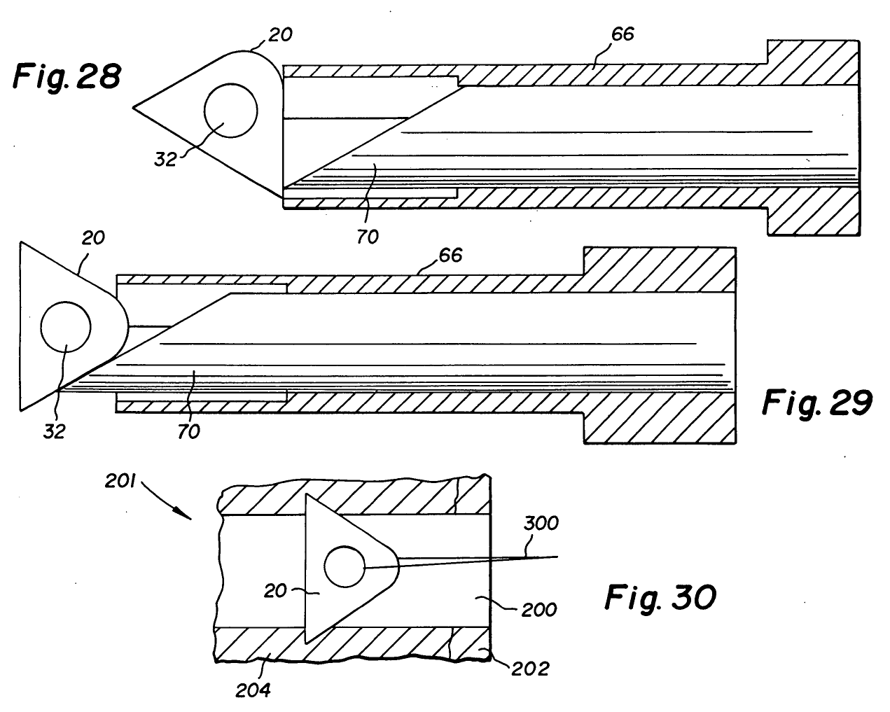

The preferred embodiment and the best mode of the suture anchor of the invention is shown in FIGS. 1-3 and the insertion tool is shown in FIGS. 4-25. The suture anchor is a sterile allograft bone suture anchor 20 with a body 22 having a distal end 24 with a radially rounded outer surface having a preferred length of 5.50 mm + or −0.10 mm when the same is used in a 3.5 mm bore hole and two tapered planar side walls 26 ending in a dome shaped or rounded proximal end 28. The general configuration of the cross section of the suture body as noted in FIG. 3 is that of a triangle. The two planar side walls 26 form an angle of approximately 60° when an axial plane extending across each planar surface 26 is extended away from the distal end 24 past the proximal end 28 to intersect forming the angle. The intersection of each side wall 26 with the distal end 24 forms a sharp end surface 30 which digs into the cancellous bone of the bore holding the suture anchor 20 firmly in place. The orientation of the suture anchor 20 in the insertion tool 50 until deposited into a bore 200 cut into the human body is sequentially shown in FIGS. 26-29. The suture anchor body 22 forms a distal end 24 with a radial outer surface having an external surface length greater than the distance from the distal end 24 to the outer surface of the proximal end 28 and greater than the diameter of the bore hole into which it is to be inserted. A throughgoing aperture 32 is formed in body 22. The aperture 32 is slightly off center and is positioned about 0.5 mm closer to the outer surface of the distal end 24 than the outer surface of the proximal end 28 so that the pulling action of the suture 300 causes the end surfaces 30 to dig the cancellous portion 204 of the bone bore 200. The aperture 32 serves to hold suture(s) 300 and allow threading of the suture anchor. Preferably, the bone anchor is manufactured from allograft cortical human bone and may be partially demineralized and alternately treated with bone morphogenic protein, bone growth factors 1-23, hylauronic acid and a phosphate buffer for quicker bone formation before the suture anchor 20 has been wedged into the cancellous portion 204 of the bone bore 200. Alternately, the suture anchor may be manufactured from a biocompatible and bioresorbable material such as xenograft bone, ceramics such as tri calcium phosphate, plastic or a biocompatible metal such as titanium or stainless steel.

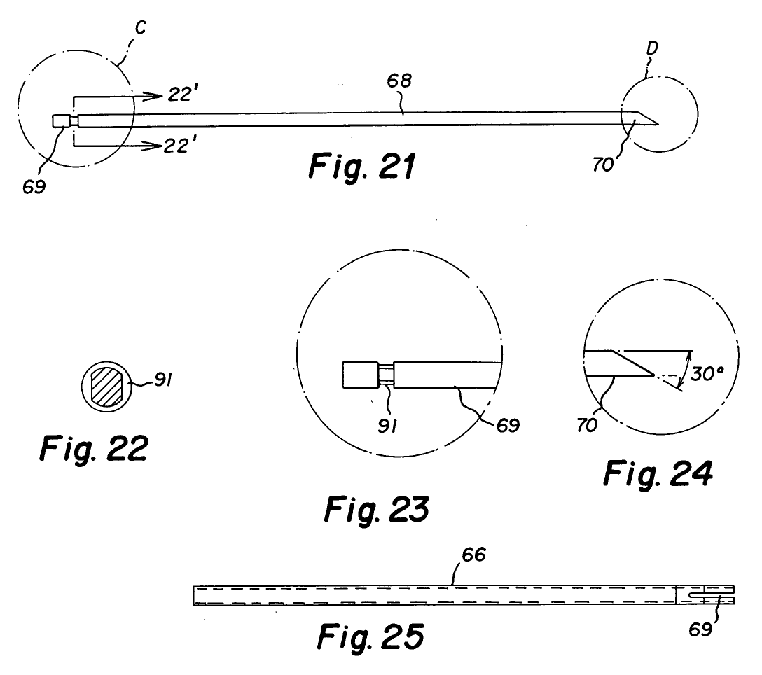

The suture anchor 20 is adapted for insertion into a hollow tube 66 of an insertion tool 50 which is shown in FIGS. 4-25. The insertion tool 50 is constructed with a curved outer surface and a generally undulating shaped handle 62 formed of polycarbonate plastic having side grips 64 formed thereon and a flared portion 65 leading to a tapered stem 67 located on the distal end. A hollow tube 66 is mounted in a bore 77 cut into the stem and flared portion 65 of the handle and terminates adjacent to a rectangular shaped cavity 63 formed in the handle 62. The hollow tube 66 is preferably constructed of 316 stainless steel. The distal end of the hollow tube 66 is provided with two parallel opposing slots 69 which are clearly shown in FIG. 25 which separate and position the suture 300 which is threaded through the aperture 32 of the suture anchor 20. The slots 69 are equally circumferentially spaced about the circumference of the tube 66 and open on the tube's distal end surface. A driver rod 68 is slidably mounted in the hollow tube 66 with the proximal end 73 of the driver rod 68 being secured to a thumb or finger activator assembly 80 (hereinafter activator assembly) by a set screw 82. The proximal end 69 of driver rod 68 has a channel 71 cut around it as seen in FIGS. 22 and 23 to receive the set screw. The driver rod 68 is preferably constructed of 316 stainless steel and has an angled distal end 70 which preferably is angled at 30° as shown in FIG. 24. The tip of end 70 engages the suture anchor 20 as the driver rod 68 is advanced by the surgeon to cam against the surface of the suture anchor 20 as is clearly shown in FIGS. 26-28. The activator assembly 80 which is mounted in cavity 63 has a body housing 81 with an outer concave finger engaging surface 82 which has a series of parallel ribs 84 formed thereon and a smooth curved distal portion 83. A suture channel 85 is cut into the finger engaging surface 82 and the curved distal portion 83 to receive and hold suture 300. The body housing 81 extends into cavity 63 formed in the handle 62. The body defines a transverse planar slide stop 88, a planar spring stop 89 and a bore 90 to hold the driver rod 68. A set screw 87 is mounted in a bore formed in body 81 transverse the bore 90 to engage the drive rod channel 91 shown in FIGS. 21-23 and hold the driver rod 68 in a fixed position in the body housing 81. A coil spring 92 is mounted over the driver rod 68 with one end engaging spring stop 89 and the other end engaging the proximal end surface of hollow tube 66 or the front end wall 78 of the handle cavity 63 positioned at the distal end of the handle cavity 63. The coil spring 92 thus constantly biases the activator assembly 80 rearward in the handle 62 so that when the activator assembly is released from driving the driver rod 68 forward in tube 66, it is driven backward to its original position against the rear wall of cavity 63. A silicone suture lock button 94 as shown in FIGS. 19 and 20 is mounted in an stepped chamber 96 formed in the handle 62 adjacent the cavity 63 and is adapted to be seated over the suture 300 to hold the suture in place during surgery. The smaller diameter of the stepped chamber 96 holds the button member 99 and serves as a stop for the foot 100 of the lock button. The suture 300 runs from the aperture 97 cut in the proximal end of the handle along channel 98 cut into the grip of the handle over channel 85 and hangs free along the barrel of the tube 66 running through slots 69.

The suture anchor 20 can be used to anchor the suture 100 in body tissue such as a piece of bone 201 having a harder outer layer of cortical bone 202 and a softer inner layer of cancellous bone 204. A fairly well defined boundary separates the outer layer cortical from the inner cancellous layer. A hole or bore 200 is provided in the bone 201 to receive and hold the anchor 20 in the bore 200.

In operation, an opening in the nature of a bore 200, which by way of a non-limiting example is 3.5 mm in diameter, is drilled through the layer of cortical bone 202 into the softer cancellous layer 204. Once the bore 200 is formed, the boring bit of the drill is removed and the suture anchor 20 is then prepared for insertion. In operation, the suture 300 is threaded through the central aperture 32 of the bone anchor 20, the bone anchor is mounted in tube 66 and the suture is pulled through the slots 67 formed on each side of the tube 66 along the barrel of the tube 66 through the respective channels in the handle and past the chamber 96. There are no knots holding the suture to the anchor as knots tend to weaken the system. The suture strands are held in place by button 94 which holds the suture strands 300 in a friction fit. The driver tool tube 66 is inserted into the bore 200 cut in the human patient and the anchor 20 is oriented through action of the driver rod 68 which is pushed forward by the push button assembly 80. The suture anchor 20 is ejected into the bore 200 and wedged into the cancellous bore portion 204 of the patient by pulling the suture 300 thus placing a pullout force on the suture anchor 20. As the suture anchor 20 is wedged into the cancellous bone 204 it becomes deeper seated and increases the peak load. The surgeon can then attach the suture opposite the suture anchor 20 to the soft tissue (not shown) and pull the soft tissue to the bone 200. Because the suture is a single piece of material, the failure strength is the suture line break strength rather than the pull out strength where two separate pieces of suture are used. Pull out of the anchor is also diminished because of the deeper seating of the suture in the bone anchor and encompassing bone mass. While various dimensions of the bore and suture anchor have been set forth it should be noted that these can vary depending upon the surgeon's preference.

In the foregoing description, the invention has been described with reference to a particular preferred embodiment, although it is to be understood that specific details as shown are merely illustrative, and the invention may be carried out in other ways without departing from the true spirit and scope of the following claims.

What we claim are:

1. A sterile suture anchor comprising:
a body with a distal end, tapered planar side walls and a dome shaped proximal end,
a cross section of said body forming a triangular shape with at least one rounded end; and
a suture aperture transversely cut through said body portion being dimensioned to hold at least one suture.

2. A sterile suture anchor according to claim 1 wherein said planar side walls form an angle of about 60°.

3. A sterile suture anchor according to claim 1 wherein said suture aperture is located nearer said proximal end than said distal end.

4. A sterile suture anchor according to claim 1 wherein said distal end has an outer surface with an area which is greater than an area of said proximal end.

5. A sterile suture anchor according to claim 1 wherein said each of said side walls intersect said distal end to form a sharp end corner.

6. A sterile suture anchor according to claim 1 wherein said suture anchor is constructed of ceramic.

7. A sterile suture anchor according to claim 6 wherein said ceramic is taken from a group consisting of tri-calcium phosphate and hydroxylapatite.

8. A sterile suture anchor as claimed in claim 1 wherein said suture anchor is constructed of allograft bone.

9. A sterile suture anchor as claimed in claim 1 wherein said suture anchor is constructed of xenograft bone.

10. A sterile suture anchor as claimed in claim 1 wherein said suture anchor is constructed of plastic.

11. A sterile suture anchor as claimed in claim 1 wherein said suture anchor is constructed of metal.

12. A sterile suture anchor as claimed in claim 11 wherein said suture anchor metal is titanium.

13. A sterile suture anchor as claimed in claim 11 wherein said suture anchor metal is stainless steel.

14. A sterile suture anchor as claimed in claim 8 wherein said allograft bone is cortical human bone.

15. A sterile suture anchor comprising:
an allograft bone body with a first distal end, said distal end having length greater than the width of a bore into which it is to be mounted, tapered planar sides extending from said distal surface and inclined toward each other so that planes formed on each side planar surface will intersect to form an angle less than 90°, said tapered planar sides terminating in a rounded proximal end with a cross section of said body having substantially triangular configuration; and
an aperture transversely cut through said bone body dimensioned to hold at least one suture located in a central section of said bone body.

16. A sterile suture anchor as claimed in claim 15 wherein said angle is about 60°.

17. A sterile suture anchor as claimed in claim 15 wherein an intersection of said distal surface and said tapered planar side walls forms sharp end edges and said distal end has a radially rounded outer surface.

18. A sterile suture anchor as claimed in claim 15 wherein the length of said distal end is greater than the distance from an outer surface of said distal end to the furthest out surfaces of said rounded proximal end.

19. A sterile suture anchor as claimed in claim 15 wherein said central aperture is circular and ranges from about 1.4 mm to about 1.8 mm in diameter.

20. A sterile suture anchor as claimed in claim 15 wherein said central aperture is nearer said first distal end than said proximal end.

21. A sterile suture anchor as claimed in claim 15 wherein said rounded proximal end is dome shaped.

22. An insertion tool for inserting a suture anchor into a substantially cylindrical hole in a live human bone and causing said suture anchor to be anchored in said live human bone, said insertion tool comprising;
a handle defining grip means and an internal cavity;
a hollow tube secured to said handle,
a driver rod slidably mounted to said tube, said driver rod having a distal end defining an angled cam which engages a suture anchor mounted in said hollow tube to rotate said suture anchor into a desired position for insertion into human tissue;
a driver assembly mounted on said handle and seated in said internal cavity, said driver assembly comprising a body, said driver rod being secured to said driver assembly body so that movement of said driver assembly causes said driver rod to be moved within said hollow tube causing said driver rod cam end to engage said suture anchor and turn said suture anchor in a predetermined orientation.

23. An insertion tool as claimed in claim 22 wherein said driver rod is spring biased opposite from the movement caused by said driver assembly.

24. An insertion tool as claimed in claim 22 wherein said cam end defines an angle of about 30°.

25. An insertion tool as claimed in claim 22 wherein said handle is constructed of plastic.

26. An insertion tool as claimed in claim 22 wherein said driver assembly body has a concave outer surface with a plurality of parallel ribs formed thereon and a channel running along the length of said body to hold a suture.

27. An insertion tool as claimed in claim 22 wherein said driver assembly body is secured to said insertion rod by screw means.

28. An insertion tool for inserting a suture anchor in a substantially cylindrical hole cut a live human bone and causing a predetermined orientation of said suture anchor in said live human bone, said insertion tool comprising;
a handle body defining grip means and an internal cavity and a suture channel along an outer surface of said handle body;
a hollow tube secured to said handle body with the proximal end of said tube leading to said handle internal cavity, a distal end of said hollow tube defining opposing slots therein;
a driver rod slidably mounted to said tube, said driver rod defining a cam end which engages a suture anchor mounted in said tube and rotates said suture anchor into a desired position for mounting in said live human bone,
a finger driver assembly mounted in said handle cavity, said finger driver assembly comprising a body, means to secure said driver rod to said body so that movement of said body causes said driver rod to be moved within said tube to engage said a suture anchor placed in said hollow tube; and
spring biasing means mounted in said handle cavity adjacent said driver rod and engaging said finger driver body to urge said driver rod back to an original position.

29. An insertion tool as claimed in claim 28 wherein said cam end defines an angle of about 30°.

30. An insertion tool as claimed in claim 28 wherein said handle defines suture holding means and a suture guide channel.

31. An insertion tool as claimed in claim 28 wherein said finger driver assembly has a concave outer surface with a plurality of parallel ribs formed thereon.

32. An insertion tool as claimed in claim 28 wherein said finger driver assembly is secured to said driver rod by a set screw which is mounted in said finger driver body and engages said driver rod.
